(19) United States
(12) Patent Application Publication
(10) Pub. No.: US 2012/0156748 A1
(43) Pub. Date: Jun. 21, 2012

(54) PROCESS FOR TREATMENT OF A LIGNOCELLULOSIC MATERIAL

(75) Inventors: Wilhelmus Maria BOS, Amsterdam (NL); Evert VAN DER HEIDE, Amsterdam (NL); Jan Gerrit DE JONG, Amsterdam (NL); Munro MACKAY, Amsterdam (NL)

(73) Assignee: SHELL OIL COMPANY, Houston, TX (US)

(21) Appl. No.: 13/330,768

(22) Filed: Dec. 20, 2011

(30) Foreign Application Priority Data
Dec. 10, 2010 (EP) EP10196052.4

(51) Int. Cl. C12P 7/10; D21C 3/04; D21C 3/00

(52) U.S. Cl. 435/165; 162/70; 162/82; 162/76

(57) ABSTRACT

A process for treatment of a lignocellulosic material comprising contacting the lignocellulosic material with a solution of chloride ions, which solution comprises a concentration of chloride ions in the range from equal to or more than 1 ppm weight to equal to or less than 350 ppm weight based on the total weight of the solution; at a temperature in the range from equal to or more than 120° C. to equal to or less than 200° C.; and at a pH in the range from equal to or more than 0.1 to equal to or less than 4.

The present application claims the benefit of EPC Patent Application Serial No. 10196052.4 filed Dec. 20, 2010, the entire disclosure of which is hereby incorporated by reference.

### FIELD OF THE INVENTION

The invention relates to a process for treatment of a lignocellulosic material.

### BACKGROUND TO THE INVENTION

With the diminishing supply of crude mineral oil, use of renewable energy sources is becoming increasingly important for the production of fuels and chemicals. These fuels and chemicals from renewable energy sources are often referred to as biofuels, respectively biochemicals. One of the advantages of using renewable energy sources is that the CO2 balance is more favourable as compared with a conventional feedstock of a mineral source.

Biofuels and/or biochemicals derived from non-edible renewable energy sources, such as lignocellulosic material, are preferred as these do not compete with food production. These biofuels and/or biochemicals are also referred to as second generation biofuels and/or biochemicals.

A wide variety of methods for converting lignocellulosic material into biofuels and/or biochemicals is available.

Larsen et al. describe in their article titled “The IBUS Process—Lignocellulosic Bioethanol Close to a Commercial Reality”, Chem. Eng. Technol. 2008, vol. 31, No. 5, pages 765-772 a so-called Integrated Biomass Utilization System (IBUS) that is said to convert biomass to bioethanol using steam and enzymes only. The article describes a pretreatment process wherein wheat straw is introduced into a pressurized reactor where the straw is heated by steam to a temperature between 180 and 200° C. with a residence time of 5-15 minutes. This is said to save substantial capital and operational costs and to reduce problems with corrosion. A drawback however is that also yields are reduced and processing time increases.

Ramos provides in their article titled “The Chemistry involved in the steam treatment of lignocellulosic materials”, Quim. Nova, Vol. 26, No. 6, pages 863-871, 2003, a review of several pretreatment methods. One of the methods described is the use of concentrated acid involving the solubilization of plant polysaccharides in 72% (w/v) sulfuric acid or 41% (w/v) hydrochloric acid at low temperatures, followed by dilution to a 3-6% (w/v) acid concentration and heating at 100-120° C. for 30-360 minutes. Although close to theoretical yields can be achieved through this technology, the process is said to involve high capital investment, acid consumption and acid recovery costs.

The Technical Report by Aden et. al. titled “Lignocellulosic Biomass to Ethanol Process Design and Economics Utilizing Co-Current Dilute Acid Prehydrolysis and Enzymatic Hydrolysis for Corn Stover”, NREL/TP-510-32438, June 2002, describes a pretreatment process using 1.1% sulfuric acid, a temperature of 190° C., a pressure of 12.1 atmosphere (177 psia) and 30% solids in the reactor. It is indicated that the material of construction of the flash tank is SS316, because additional acid resistance at temperatures higher than 100° C. is necessary.

### SUMMARY OF THE INVENTION

It would be advantageous to provide a cheaper, but still efficient process for the treatment of lignocellulosic material. For example it would be advantageous to provide a process for the treatment of lignocellulosic material which would allow for high yields whilst avoiding the need to use equipment with a higher acid resistance. Such a cheap but efficient process, which allows for high yields whilst avoiding expensive equipment, has now been found.

Accordingly, in one embodiment, provides a process for treatment of a lignocellulosic material comprising contacting the lignocellulosic material with a solution of chloride ions, which solution comprises a concentration of chloride ions in the range from equal to or more than 1 ppm weight to equal to or less than 350 ppm weight based on the total weight of the solution; at a temperature in the range from equal to or more than 120° C. to equal to or less than 200° C.; and at a pH in the range from equal to or more than 0.1 to equal to or less than 4.

In another embodiment, provides a process for reducing chloride content in a chloride containing lignocellulosic material comprising the steps of

a) mixing the chloride containing lignocellulosic material with an aqueous solution to provide an aqueous slurry of lignocellulosic material;

b) passing (forwarding) the aqueous slurry of lignocellulosic material to a separation device to provide a chloride-depleted lignocellulosic material and a chloride-enriched aqueous solution;

c) separating the chloride-depleted lignocellulosic material and a chloride-enriched aqueous solution.

Such a process advantageously allows for a convenient method for providing the solution of chloride ions used in the process for treatment of a lignocellulosic material mentioned above.

### BRIEF DESCRIPTION OF THE DRAWINGS

The invention has been illustrated by the following non-limiting FIGURE:

FIG. 1, illustrating an embodiment of the invention wherein the chloride content of a lignocellulosic material is reduced to conveniently provide a solution of chloride ions.

### DETAILED DESCRIPTION OF THE INVENTION

The process according to the invention advantageously reduces and in many cases even eliminates corrosion of stainless steel construction materials, thereby allowing the use of less expensive stainless steel and/or less expensive stainless steel alloys as construction material. At the same time high yields, for example high conversions of lignocellulosic material, can still be achieved in the process. In an embodiment of the present invention, provides a process comprising contacting a construction material, which construction material comprises stainless steel and/or a stainless steel alloy, with a solution of chloride ions, which solution comprises a concentration of chloride ions in the range from equal to or more than 1 ppm weight to equal to or less than 100 ppm weight, based on the total weight of the solution; at a temperature in the range from equal to or more than 120° C. to equal to or less than 170° C.; and at a pH in the range from equal to or more than 1.5 to equal to or less than 4.

By lignocellulosic material is herein understood a material containing cellulose, hemicellulose and lignin.

In addition, the process according to the invention provides a treated lignocellulosic material which is very suitable for further processing, for example by enzymatic hydrolysis of the pretreated lignocellulosic material or components thereof.

The lignocellulosic material may be obtained from a wide variety of sources, including for example plants, forestry residues, agricultural residues, herbaceous material, municipal solid wastes, waste and recycled paper, pulp and paper mill residues, sugar processing residues and/or combinations of one or more of the above.

The lignocellulosic material can comprise for example, corn stover, soybean stover, corn cobs, corn fibre, straw (including cereal straws such as wheat, barley, rye and/or oat straw), bagasse, beet pulp, miscanthus, sorghum residue, rice straw, rice hulls, oat hulls, grasses (including switch grass), bamboo, water hyacinth, wood and wood-related materials (including hardwood, hardwood chips, hardwood pulp, softwood, softwood chips, softwood pulp and/or sawdust), waste paper and/or a combination of one or more of these feedstocks.

Prior to treatment according to the process of the invention, the lignocellulosic material can be washed and/or reduced in particle size. The particle size reduction may for example include grinding, chopping, crushing or debarking of a lignocellulosic material. Preferably the particle size of the lignocellulosic material is reduced to a particle size in the range from equal to or more than 5 micron to equal to or less than 5 cm, more preferably in the range from 2 mm to 10 mm.

Thus, in some embodiments, providing lignocellulosic material can comprise harvesting a lignocelluloses-containing plant such as, for example, a hardwood or softwood tree. The tree can be subjected to debarking, chopping to wood chips of desirable thickness, and washing to remove any residual soil, dirt and the like.

The lignocellulosic material is contacted with a solution of chloride ions (i.e. Cl⁻ ions). The solution of chloride ions comprises a concentration of chloride ions in the range from equal to or more than 1 ppm weight to equal to or less than 350 ppm weight based on the total weight of the solution. In a preferred embodiment the solution of chloride ions is an aqueous solution of chloride ions, which aqueous solution contains equal to or more than 1 ppm weight to equal to or less than 300 ppm weight of chloride ions based on the total weight of the solution.

More preferably the concentration of chloride ions in the (preferably aqueous) solution is equal to or more than 2 ppm weight, yet more preferably equal to or more than 5 ppm weight, even more preferably equal to or more than 10 ppm weight, still more preferably equal to or more than 15 ppm weight, and most preferably equal to or more than 20 ppm weight, and/or preferably equal to or less than 200 ppm weight, more preferably equal to or less than 150 ppm weight, yet more preferably equal to or less than 100 ppm weight, even more preferably equal to or less than 90 ppm weight, still more preferably equal to or less than 80 ppm weight, and most preferably equal to or less than 70 ppm weight, based on the total weight of the solution. Hence in a preferred embodiment the concentration of chloride ions in the (preferably aqueous) solution lies in the range from equal to or more than 2 ppm weight to equal to or less than 150 ppm weight, more preferably in the range from equal to or more than 5 ppm weight to equal to or less than 100 ppm weight and most preferably in the range from equal to or more than 10 ppm weight to equal to or less than 90 ppm weight.

In a preferred embodiment the combined effects of the concentration of chloride ions and temperature are taken into account for the process of the invention. Preferably the log of the concentration of chloride ions in the (preferably aqueous) solution is equal to or more than 1. More preferably the log of the concentration of chloride ions in the solution is equal to or less than the log of the concentration of chloride ions as calculated according to formula I (herein also referred to as boundary log concentration of chloride ions);

log [Cl⁻]_{(boundary)}=−0.011*T+3.7 (formula I)

wherein T represents the temperature in degrees Celsius at which the contacting with the lignocellulosic material is taking place.

The solution of chloride ions can be provided in any manner known by the skilled person to be suitable for such a purpose. For example it may be provided by solving a suitable amount of a chloride salt (such as for example potassium chloride, sodium chloride, calcium chloride and/or mixtures thereof) or it may be formed by solving a suitable amount of a chloride acid, such as for example hydrochloric acid (HCl).

In one embodiment the desired concentration of chloride ions may be suitably reached by addition of a chloride salt or chloride acid to a, preferably aqueous, solution that is essentially free of chloride ions.

In another embodiment the desired concentration of chloride ions may be reached by dilution of a solution comprising a higher concentration of chloride ions.

In another embodiment the desired concentration of chloride ions may be suitable provided at least in part by dissolving chloride ions present in the lignocellulosic material in a solution such that a solution with the desired concentration of chloride ions is formed.

In still another embodiment the solution with the desired concentration of chloride ions may be suitably provided by at least partly recycling of a chloride ion containing solution obtained elsewhere in the process, for example a chloride ion containing solution obtained after separation of a first liquid stream from a product mixture provided by the process of the invention; or a chloride ion containing solution obtained after reducing the chloride content of a lignocellulosic material.

The lignocellulosic material is contacted with the solution of chloride ions at a temperature in the range from equal to or more than 120° C. to equal to or less than 200° C.

More preferably the temperature is equal to or more than 130° C., still more preferably equal to or more than 140° C. and/or preferably equal to or less than 190° C., more preferably equal to or less than 180° C., still more preferably equal to or less than 170° C., most preferably equal to or less than 160° C. Hence, in a preferred embodiment the lignocellulosic material is contacted with the solution of chloride ions at a temperature in the range from equal to or more than 130° C. to equal to or less than 190° C., more preferably in the range from equal to or more than 140° C. to equal to or less than 170° C.

In a suitable embodiment the lignocellulosic material and/or the solution of chloride ions may be preheated before being contacted with each other. If desired such preheating can be carried out at least partially via heat exchange with the effluent of the process according to the invention or the effluent of subsequent processing steps as described herein below.

The lignocellulosic material is contacted with the solution of chloride ions at a pH in the range from equal to or more than 0.1 to equal to or less than 4.

Preferably the pH is equal to or more than 1.0, more preferably equal to or more than 1.2, still more preferably equal to or more than 1.5 and most preferably equal to or more than 1.8 and/or preferably equal to or less than 3, more preferably equal to or less than 2.8 and most preferably equal to or less than 2.6. Hence in a preferred embodiment the lignocellulosic material is contacted with the solution of chloride ions at a pH is in the range from equal to or more than 1.5 to equal to or less than 2.8, more preferably in the range from equal to or more than 1.8 to equal to or less than 2.6.

In some cases the pH may slightly vary during the reaction of the lignocellulosic material with the solution of chloride ions, in such cases the pH at the start of the contacting of the lignocellulosic material with the solution of chloride ions—sometimes referred to as pre-reaction pH—is preferably within the range as indicated above. The pH will then be the pH of the solution of chloride ions. In such a case, the pH of the solution of chloride ions is preferably within the ranges as indicated above.

The claimed range of pH can suitably be reached by addition of a sufficient amount of acid to a neutral or basic solution or by dilution of an acid solution.

In a preferred embodiment therefore the solution of chloride ions comprises an aqueous solution of chloride ions that contains also an acid. The acid preferably comprises one or more weak organic acids, more preferably the acid comprises an acid chosen from the group consisting of formic acid, sulphuric acid, acetic acid, citric acid, levulinic acid and/or mixtures thereof.

An aqueous solution of chloride ions and acid preferably contains the acid in a concentration of equal to or less than 20 wt % acid, preferably equal to or less than 10 wt %, more preferably equal to or less than 5 wt %, even more preferably equal to or less than 2 wt % acid, and most preferably equal to or less than 1 wt % acid based on the total weight of the aqueous solution. For practical purposes, the acid concentration is preferably equal to or more than 0.01 wt % acid, more preferably equal to or more than 0.05 wt % acid, and still more preferably equal to or more than 0.1 wt % acid, based on the total weight of the aqueous solution.

Hence in a preferred embodiment the lignocellulosic material is contacted with a solution of chloride ions and an additional acid, where in the additional acid is chosen from the group consisting of formic acid, sulphuric acid, acetic acid, citric acid, levulinic acid and/or mixtures thereof, and wherein the additional acid is present in a concentration in the range from equal to or more than 0.01 wt % acid to equal to or less than 20 wt % of acid, more preferably in the range from equal to or more than 0.05 wt % to equal to or less than 10 wt % of acid, most preferably in the range from equal to or more than 0.1 wt % acid to equal to or less than 5 wt % acid, based on the total weight of the aqueous solution.

The treatment according to the process of the invention can be carried out in a continuous, semi-batch or batch mode. In a preferred embodiment contacting time of the lignocellulosic material with the solution of chloride ions lies in the range of from equal to more than 1 minute, more preferably from equal to or more than 5 minutes, still more preferably from equal to or more than 10 minutes to equal to or less than 2 hours, more preferably equal to or less than 1.5 hour, still more preferably equal to or less than 1 hour.

In a preferred embodiment the combined effects of the pH and temperature are taken into account for the process of the invention. Preferably the pH at which the lignocellulosic material is contacted with the solution of chloride ions is equal to or more than a pH as calculated according to formula II, herein also referred to as boundary pH;

pH_{(boundary)}=log(exp(T−Tref)/14.75)−0.5 (formula II)

wherein T represents the temperature in degrees Celsius at which the contacting is taking place; and wherein Tref represents a reference temperature of 100° C.

In cases where the pH may slightly vary during the reaction, the pH at the start of the reaction (sometimes referred to as pre-reaction pH) is preferably within the range as indicated above.

In a further preferred embodiment the combined effects of contacting time and temperature are taken into account for the process of the invention. In the process according to the invention the lignocellulosic material is therefore preferably contacted with the solution of chloride ions at a severity factor log(Ro) from equal to or more than 2.5, more preferably from equal to or more than 3, to equal to or less than 4.5, more preferably to equal to or less than 4. The severity factor log(Ro) at a specific contacting time and at a specific temperature can be calculated according to formula III, wherein:

log(Ro)=log(t·exp(T−T(ref))/14.75) formula (III)

wherein log(Ro) represents the so-called severity factor; wherein t represents time in minutes at which the contacting is taking place and T represents the temperature in degrees Celsius at which the contacting is taking place; and wherein T(ref) represents a reference temperature of 100° C.

The lignocellulosic material-to-solution weight ratio (i.e. the weight ratio of solid to solvent) is preferably in the range of from 2-to-1 (2:1) to 1-to-10 (1:10), more preferably in the range of from 1-to-3 (1:3) to 1-to-8 (1:8), most preferably in the range from 1-to-3 (1:3) to 1-to-5 (1:5).

The process of the invention is preferably performed at a pressure where water at the temperature applied does not boil yet. For practical purposes the pressure preferably lies in the range from equal to or more than an atmospheric pressure of about 1 bar absolute (about 0.1 MPa) to equal to or less than 15 bar absolute (1.5 MPa), more preferably a pressure of equal to or less than 10 bar absolute (1 MPa). Preferably the process is carried out at atmospheric pressure of about 1 bar absolute (i.e. about 0.1 MPa))

The process according to the invention may be carried out in any type of reactor known to the skilled person to be suitable for this purpose. The reactor is preferably a batch reactor, a CSTR reactor, or a plug flow reactor or a slurry reactor having an arrangement to move the lignocellulosic material mechanically. The reactor may comprise mechanical means for the propagation or displacement of the lignocellulosic material. For example, the reactor may comprise a stirrer or a screw.

In a preferred embodiment the reactor contains a lignocellulosic material and in operation an aqueous solution is sprayed onto the lignocellulosic material such that in-situ an aqueous solution of chloride ions is formed.

Preferably the reaction (due to contacting the lignocellulosic material and the solution of chloride ions) is carried out whilst the lignocellulosic material is forwarded through a so-called screw-press.

The process of the invention advantageously allows one to use cheaper construction materials, preferably construction materials comprising stainless steel and/or stainless steel alloys (also referred to herein as stainless steel construction material(s) and/or stainless steel alloy construction material(s)). Therefore, although the process can be carried out in one or more reactor(s) of carbon steel, in a preferred embodiment the lignocellulosic material is contacted with the solution of chloride ions in one or more reactor(s) comprising one or more stainless steel construction material(s) and/or stainless steel alloy construction material(s). The one or more stainless steel construction material(s) and/or stainless steel alloy construction material(s) can suitably be in direct contact with the solution of chloride ions. In a preferred embodiment the process according to the invention is carried out in one or more reactor(s) comprising stainless steel construction material(s) and/or stainless steel alloy construction material(s), which material(s) contain equal to or less than 20.0 mol % chromium, more preferably equal to or less than 19.5 mol % chromium, still more preferably equal to or less than 19.0 mol % chromium, most preferably equal to or less than 18.5 mol % chromium; and/or equal to or less than 18.0 mol % nickel, more preferably equal to or less than 17.0 mol % nickel, still more preferably equal to or less than 16.0 mol % nickel, most preferably equal to or less than 15.0 mol % nickel. For practical purposes the stainless steel construction material(s) and/or stainless steel alloy construction material(s) may contain preferably equal to or more than 10.0 mol % chromium, more preferably equal to or more than 11.0 mol % chromium, most preferably equal to or more than 12.0 mol % chromium; and/or equal to or more than 7.0 mol % nickel, more preferably equal to or more than 8.0 mol % nickel, most preferably equal to or more than 9.0 mol % nickel. Examples of preferred types of stainless steel construction material(s) and/or stainless steel alloy construction material(s) for the vessel in which the process according to the invention is carried out include 254SMO (UNS S31254), 654SMO, SAF2507, 904L, Alloy 33 (30 mol % Ni), SAE-316 (UNS S31600), SAE-316L (UNS S31603), SAE-316LN (UNS S31653), SAE-321H (UNS S32109), SAE-321 (UNS S32100), SAE-305 (UNS S30500), SAE-304H (UNS S30409), SAE-317L (UNS S31703), SAE-321 (UNS S32100), SAE-304 (UNS S30400), SAE-304LN (UNS S30453), SAE-304L (UNS S30403) and/or SAE-301 (UNS S30100) and/or combinations thereof. Most preferably the stainless steel construction material(s) and/or stainless steel alloy construction material(s) comprises SAE-316 (UNS31600), SAE-316L (UNS S31603) and/or 254 SMO. Of these SAE-316L (UNS S31603) is preferred over SAE-316 (UNS31600). Advantageously 316L, the low carbon version of 316, is less susceptible to sensitisation (also referred to as grain boundary carbide precipitation), whilst there is commonly no appreciable price difference between 316 and 316L stainless steel.

The process according to the invention can be a process which is essentially entirely, that is during essentially the whole of the contacting time, carried out at the pH, temperature and concentration of chloride ions as specified herein; or a process which is at least partly, that is during part of the contacting time, carried out at the pH, temperature and concentration of chloride ions as specified herein. That is, during the process one can move in and out of the area as specified by the temperature, pH and chloride ion concentration boundaries provided herein. As long as at least a substantial part of the process is carried out according to the invention, the advantages of the process, including decreased corrosion of stainless steel construction materials may still be achieved. In a preferred embodiment, however, at least 25%, more preferably at least 50% and most preferably at least 75% of the time the lignocellulosic material is actively contacted with the solution of chloride ions, such contacting is at a pH and temperature as specified herein. By actively contacting is herein understood contacting at conditions which are essentially effective to convert the lignocellulosic material into cellulose, hemicellulose and/or lignin. The contacting is considered inactive when pH is such, for example above 4, and temperature is such, for example below 120° C., that essentially no lignocellulosic material is converted.

In an especially preferred embodiment the process according to the invention comprises contacting the lignocellulosic material with a solution of chloride ions which solution comprises a concentration of chloride ions in the range from equal to or more than 1 ppm weight to equal to or less than 100 ppm weight, preferably equal to or less than 90 ppm weight, more preferably equal to or less than 80 ppm weight, even more preferably equal to or less than 70 ppm weight, most preferably equal to or less than 60 ppm weight based on the total weight of the solution; at a temperature in the range from equal to or more than 120° C. to equal to or less than 170° C., preferably equal to or less than 150° C.; and a pH in the range from equal to or more than 1.5, more preferably equal to or more than 2.0 and most preferably equal to or more than 2.5 to equal to or less than 4, preferably equal to or less than 3, preferably in a vessel comprising one or more construction material(s) of a stainless steel and/or construction materials of a stainless steel alloy.

Without wishing to be bound by any kind of theory, it is believed that such a process essentially eliminates corrosion of the stainless steel construction material(s) and/or stainless steel alloy construction material(s).

The skilled person will further also realize that the present invention provides a process comprising contacting a construction material comprising stainless steel and/or a stainless steel alloy, preferably as described above, with a solution of chloride ions, which solution comprises a concentration of chloride ions in the range from equal to or more than 1 ppm weight to equal to or less than 100 ppm weight, preferably equal to or less than 90 ppm weight, more preferably equal to or less than 80 ppm weight, even more preferably equal to or less than 70 ppm weight, most preferably equal to or less than 60 ppm weight based on the total weight of the solution; at a temperature in the range from equal to or more than 120° C. to equal to or less than 170° C., preferably equal to or less than 150° C.; and a pH in the range from equal to or more than 1.5, more preferably equal to or more than 2.0 and most preferably equal to or more than 2.5 to equal to or less than 4, preferably equal to or less than 3. Preferences for such a process are as described above.

The process of treatment according to the invention suitably provides a product mixture containing hemicellulose and/or sugars and/or cellulose and/or lignin.

In a preferred embodiment such product mixture may be separated into a first liquid stream comprising hemicellulose and/or sugars and a second stream comprising lignin and/or cellulose. The first liquid stream preferably comprises water; chloride ions; acid; hemicellulose that is optionally at least partly hydrolysed; and optionally sugars such as xylose. The second stream preferably comprising cellulose and, optionally destructured, lignin. In a preferred embodiment the second stream comprises solids, most preferably in the form of a solid residue. The first liquid stream and the second stream can be separated using separation techniques known to the skilled person to be suitable for this purpose. For example the product mixture may suitably be separated into the first liquid stream and the second stream via filtration, centrifugation, settling, and/or via the use of cyclones and or one or more screw presses.

Preferably the separation involves removal of the first liquid stream from the second stream by pressing the liquid out from a slurry resulting in the formation of two separate streams; a first liquid stream and a second solid comprising stream.

The process according to the invention advantageously also allows the use of any equipment for separation of the first liquid stream and the second stream that comprises stainless steel construction material(s) and/or stainless steel alloy construction material(s) of the types as described above.

In a preferred embodiment at least part of the obtained first liquid stream is recycled and contacted with fresh lignocellulosic material in the treatment process of the invention. This advantageously allows for higher concentrations of hydrolysed hemicellulose (in specific of xylose) to be built up in the liquid stream.

At least part of the product mixture can advantageously be converted into a biofuel and/or a biochemical.

Cellulose and/or lignin present in the second stream after separation may preferably be subjected to further processing to obtain lignin and cellulose in purer forms which can be used for the production of chemicals and fuels. In an especially preferred embodiment the second stream is washed one or more times before it is processed in a next step.

In a preferred embodiment cellulose recovered from the second stream is at least partly delignified and subsequently used in industrial cellulose applications, with or without drying, or subjected to further processing to either modify the cellulose or convert it into glucose. The cellulose preferably may be processed into paper products by any convenient methods, as those disclosed in Macdonald, Papermaking and Paperboard Making, Vol. 3, TS 1048.J66, 1969. The cellulose may also useful as fluff pulp, which is commonly used in absorbent applications such as diapers and consumer wipes.

In another preferred embodiment cellulose obtained from the second stream is enzymatically hydrolyzed to sugars such as for example glucose and soluble glucose containing oligomers. Such sugars can advantageously be converted into bioethanol, a valuable biofuel component. Enzymes suitable for such enzymatic hydrolysis of cellulose include cellulases.

In a still further preferred embodiment cellulose obtained from the second stream is catalytically or thermally converted to various organic acids, alcohols and other materials.

Lignin can be used as a fuel, it can act as an oxygenated component in liquid fuels and/or it can be used as a chemical precursor for producing lignin derivatives, for example polyphenolic polymers such as Bakelite.

Hemicellulose and/or sugars obtained from the first liquid stream can advantageously be converted in one or more steps to furfural or one or more hydrocarbons that are suitable as a biofuel or biochemical component. Or, in an alternative embodiment hemicellulose and/or sugars obtained from the first liquid stream may be enzymatically converted to a biofuel (including but not limited to bioethanol) or biochemical component.

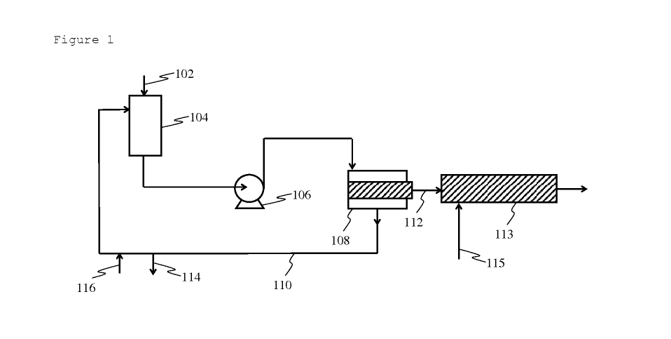

FIG. 1 illustrates an embodiment of the invention wherein the chloride content of a lignocellulosic material is reduced to provide a solution of the claimed concentration of chloride ions. In FIG. 1 a lignocellulosic material containing 2000 ppm weight chloride ions (102) is provided to a mixing vessel (104), where it is mixed with a solution of water and acid of a specific pH. The mixture of lignocellulosic material and solution is forwarded to pump (106) to bring it up to a pressure of 10-15 bar absolute (1.0-1.5 MPa) and subsequently forwarded to a screw press (108). In the screw press (108) liquid is pressed out (110) and a lignocellulosic material with a reduced content of chloride ions (112) is obtained. The lignocellulosic material with the reduced content of chloride ions (112) can be fed to a reactor (113) for contacting with a solution of chloride ions (115). The solution of chloride ions (115) may optionally be at least partly obtained from the liquid (110) obtained from the screw press (108). Suitably the solution of water and acid of a specific pH can be partly bleeded for cleaning (114) and fresh solution is provided via a make-up stream (116).

### EXAMPLES

The invention will now be further illustrated by means of the following non-limiting examples.

### Examples 1-4

### Rotating Wing Test

The Rotating wing test was used for assessing pitting corrosion on stainless steel under the conditions of the present invention. In this test, coupons of material to test, were mounted in a rotating wing, to simulate a high liquid velocity. The weight loss of the coupons as result of the test was determined and microscopic investigation of the surface of the coupons was performed. The experiments were performed in a 1.5 litre glass, double-walled Buechi autoclave. The temperature was controlled by pumping hot thermo stated oil between the double walls of the autoclave. The experiments were performed at a pressure of 6 bar gauge (0.7 MPa) and a temperature of 150 and 165° C. in the autoclave lid, venting pipes, through which gas can be introduced and released, a safety valve and a pressure gauge were present. A Pt100 thermo element for recording the temperature was also mounted through the lid.

The wing was rotated with a speed of 400 rpm. Combined with the diameter of the wing (62 mm) this resulted in a linear velocity for the coupons of 1.3 m/s.

The stainless steels subjected to the rotating wing tests were AISI 316L (UNS S31603) and 254SMO (UNS S31254). AISI 316L is the cheaper material, containing about 17.24 mol % of Chromium and 11.11 mol % of nickel. 254SMO has a higher corrosion resistance, containing about 19.91 mol % Chromium and 18.12 wt % nickel.

The experimental procedure was as follows: The test medium with the pH and chloride content as specified in table 1 was prepared by weighing a somewhat smaller amount than calculated of 10% m/m sulphuric acid (prepared from 98 wt % sulphuric acid) and NaCl in 1400 g demineralised water. The pH was measured and adjusted by adding diluted sulphuric acid. Finally, water was added up to a total weight of 1500 g test solution.

The steel coupon was ground with P400 grit paper, rinsed with water and acetone, dried, weighed and mounted in the wing after microscopic inspection. The glass autoclave vessel was mounted and the liquid phase added. After closing the autoclave, the stirring and heating was started and the content was purged with air until a temperature of 90° C. was attained after about 1 hour. Subsequently, the off gas valve of the autoclave was closed. The gas supply was closed after reaching an air pressure of 1 bar gauge (0.2 MPa). Heating of the autoclave continued until the test temperature was attained. The exposure time varied from 48 to 163 hours (as indicated in table 1). After the planned exposure time, heating was switched off, allowing the autoclave to cool down before opening. The coupons were cleaned by brushing with an abrasive cleaning agent, rinsed with water and acetone, dried and weighed. The weight loss and exposure time were used to calculate the overall corrosion rate. A microscopic examination up to a magnitude of 40× was performed to determine the type and degree of the attack by corrosion.

The results of the tests for pitting corrosion under passive condition are given in Table 1. For AISI 316L only limited pitting corrosion was found on the leading edge of the coupon after the test with 100 ppm weight chloride at 150 and 165° C. Tests in the condition with 500 ppm weight Chloride show severe pitting corrosion on all sides of the 316 type coupons at both temperatures. The coupons of 254SMO did not suffer from pitting corrosion in all performed tests.

TABLE 1
Pitting corrosion with rotating wing test

| | | | | | Microscopic | Microscopic |
|---|---|---|---|---|---|---|
| | | | | Testing | examination of | examination |
| | T | | [Cl−] | time | coupon | of coupon |
| Example | (° C.) | pH | ppmw | (hours) | AISI 316L | 254SMO |
| 1 | 150 | 3.0 | 100 | 114.5 | limited pitting | No pitting |
| | | | | | corrosion | corrosion |
| 2 | 165 | 2.5 | 100 | 163 | limited pitting | No pitting |
| | | | | | corrosion | corrosion |
| 3 | 150 | 2.5 | 500 | 48 | severe pitting | No pitting |
| | | | | | corrosion | corrosion |
| 4 | 165 | 2.5 | 500 | 115 | severe pitting | No pitting |
| | | | | | corrosion | corrosion |

### Example 5-14

### Conversion of Lignocellulosic Material

In order to assess conversion, 10 g of a lignocellulosic material as listed in table 2 and 100 ml of an aqueous H2SO4 solution with a pH (pre-reaction) as listed in table 2 were placed in a 300 ml autoclave (Parr) and heated during about 30 minutes to the desired temperature as listed in table 2, where treatment was assumed to start (t=0). After reaching the pre-set treatment time as listed in table 2, the treatment was terminated by forced water cooling, the autoclave was opened, liquid was separated from the solid residue by filtration and the pH (post-reaction) was measured. The remaining solid residue was washed twice with water and dried at 50° C. and 200 mbar (0.02 MPa) pressure to constant weight. The weight of the solid residue after washing and drying was determined (i.e the weight of the solid residue on dry matter basis) and the difference in weight on dry matter basis between the solid residue and the feed of lignocellulosic material (10 g) was used to calculate the conversion of the lignocellulosic material (wt %).

TABLE 2
Conversion of lignocellulosic material

| | Ligno- | | | | | | |
|---|---|---|---|---|---|---|---|
| | cellulosic | pH | T | [Cl−] | Time | pH | Conversion |
| Ex. | material | (pre) | (° C.) | (mg/kg) | (min.) | (post) | (wt %) |
| 5 | birch | 1.8 | 150 | 9.9 | 120 | 2.5 | 33.31 |
| 6 | birch | 2 | 170 | 8.7 | 120 | 3.1 | 35.76 |
| 7 | birch | 2.5 | 150 | 7.2 | 120 | 3.6 | 30.34 |
| 8 | straw | 2.5 | 150 | 327 | 120 | 4.5 | 24.70 |
| 9 | straw | 2.5 | 170 | 336 | 120 | 3.9 | 25.59 |
| 10 | birch | 2.5 | 170 | 16.7 | 120 | 3.4 | 33.87 |
| 11 | straw | 1.5 | 170 | 332 | 120 | 2.5 | 37.84 |
| 12 | birch | 1.9 | 170 | 19.4 | 120 | 2.9 | 35.43 |
| 13 | birch | 1.9 | 150 | 7.5 | 120 | 2.8 | 31.88 |
| 14 | straw | 1.5 | 150 | 314 | 120 | 2.4 | 35.57 |

What is claimed is:

1. A process for treatment of a lignocellulosic material comprising contacting the lignocellulosic material with a solution of chloride ions, which solution comprises a concentration of chloride ions in the range from equal to or more than 1 ppm weight to equal to or less than 350 ppm weight based on the total weight of the solution; at a temperature in the range from equal to or more than 120° C. to equal to or less than 200° C.; and at a pH in the range from equal to or more than 0.1 to equal to or less than 4.

2. The process of claim 1 wherein the chloride ions are provided at least in part by dissolving chloride ions present in the lignocellulosic material in the solution.

3. The process of claim 1 wherein the acid comprises formic acid, sulfuric acid, acetic acid, citric acid, levulinic acid and/or mixtures thereof.

4. The process of claim 3 wherein the chloride ions are provided at least in part by dissolving chloride ions present in the lignocellulosic material in the solution.

5. The process of claim 1 wherein the pH is equal to or more than a pH as calculated according to formula II,
pH_{(boundary)}=log(exp(T−Tref)/14.75)−0.5 (formula II)
wherein T represents the temperature in degrees Celsius at which the contacting is taking place; and wherein Tref represents a reference temperature of 100° C.

6. The process of claim 1 wherein the pH is determined at the start of the reaction.

7. The process of clam 1 wherein the lignocellulosic material is contacted with the solution of chloride ions at a severity factor log(Ro) from equal to or more than 2.5 to equal to or less than 4.5 and wherein the severity factor log(Ro) is calculated according to formula III:
log(Ro)=log(t·exp(T−T(ref))/14.75) formula (III)
wherein log(Ro) represents the severity factor, t represents time in minutes, T represents the temperature in degrees Celsius at which the contacting is taking place, and Tref represents a reference temperature of 100° C.

8. The process of claim 1 wherein the lignocellulosic material is contacted with the solution of chloride ions in the presence of a construction material comprising stainless steel and/or a stainless steel alloy.

9. The process of claim 1 wherein the lignocellulosic material is contacted with the solution of chloride ions in at least one reactor comprising stainless steel construction material and/or stainless steel alloy material, which construction material(s) are in direct contact with the solution of chloride ions.

10. The process of claim 9, wherein the stainless steel or stainless steel alloy contains equal to or less than 20.0 mol % chromium; and/or equal to or less than 18.0 mol % nickel.

11. The process of claim 1 wherein the process provides a product mixture containing hemicellulose and/or sugars and/or cellulose and/or lignin.

12. The process of claim 11, wherein the product mixture is separated into a first liquid stream comprising hemicellulose and/or sugars and a second stream comprising lignin and cellulose using separation equipment that comprises stainless steel and/or a stainless steel alloy.

13. The process of claim 11 wherein at least part of the product mixture is converted into a biofuel and/or a biochemical.

14. The process of claim 11 wherein cellulose is obtained and subsequently enzymatically hydrolyzed to sugars, which sugars are converted to bioethanol.

15. A process for reducing chloride content in a chloride containing lignocellulosic material comprising the steps of
a) mixing the chloride containing lignocellulosic material with an aqueous solution to provide an aqueous slurry of lignocellulosic material;
b) passing the aqueous slurry of lignocellulosic material to a separation device to provide a chloride-depleted lignocellulosic material and a chloride-enriched aqueous solution;
c) separating the chloride-depleted lignocellulosic material and a chloride-enriched aqueous solution.

16. The process of claim 15 wherein the aqueous solution comprises an aqueous solution of chloride ions comprising a concentration of chloride ions in the range from equal to or more than 1 to equal to or less than 350 ppm weight, based on the total weight of the solution.
